(19) United States
(12) Patent Application Publication
(10) Pub. No.: US 2021/0254138 A1
(43) Pub. Date: Aug. 19, 2021

(54) SIALIC ACID TRANSPORTER PROTEINS AS BIOMARKERS AND DRUG TARGETS

(71) Applicant: Quadram Institute Bioscience, Norwich, Norfolk (GB)

(72) Inventors: Nathalie Juge, Norwich, Norfolk (GB); Andrew Bell, Norwich, Norfolk (GB); John Walshaw, Norwich, Norfolk (GB)

(73) Assignee: Quadram Institute Bioscience, Norwich, Norfolk (GB)

(21) Appl. No.: 17/252,619

(22) PCT Filed: Jun. 17, 2019
(86) PCT No.: PCT/GB2019/051691

§ 371 Date: Dec. 15, 2020

(30) Foreign Application Priority Data
Jun. 15, 2018 (GB) 1809877.2

(51) Int. Cl. C12Q 1/689; C12Q 1/04; C12Q 1/686; A61K 45/06

(52) U.S. Cl. CPC C12Q 1/689; A61K 45/06; C12Q 1/686; C12Q 1/04

(57) ABSTRACT

A method of identifying, monitoring and/or diagnosing mucosal bacterial presence or infection, said method including the step of detecting at least part of a sialic acid transporter protein encoded by Ruminococcus gnavus (R. gnavus) ATCC 29149 Nan cluster. In addition, a method of inhibition of the growth of bacterium, said method including the step of inhibition of a sialic acid transporter protein is included.

The present invention relates to a biomarker and/or drug target for mucosal diseases.

Although the following description refers exclusively to biomarker/drug target for inflammatory bowel disease (IBD), the person skilled in the art will appreciate that the invention can be used as a biomarker and/or drug target for a number of mucosal diseases and is not limited to a faecal biomarker or drug target for inflammatory bowel diseases.

IBD which includes Crohn's disease (CD) and ulcerative colitis (UC) is characterised by chronic inflammation of the gastrointestinal (GI) tract which is associated with changes in the gut microbiome. Characteristics of faecal microbiota are attractive biomarkers in IBD because they provide a non-invasive way to monitor changes in the intestinal environment associated with mucosal inflammation. Microbial biomarkers do not necessarily play a causative role in disease. The appearance or disappearance of a microbial group may rather be related to how well the organism can compete in the altered intestinal environment in disease.

IBD is characterised by changes in mucin glycosylation (i.e. decrease in complex mucin glycan, increased sialylation) and dysbiosis (changes in microbiota composition). Several metagenomics studies have shown a disproportionate increase in certain mucosa-associated bacteria such as Ruminococcus gnavus in IBD. As stated above faecal microbiota are attractive biomarkers because they provide a non-invasive way to monitor changes occurring at the mucosal interface.

The use of microbe signatures as biomarkers is currently hampered by the phylogenetic resolution achievable by 16S rRNA which does not allow distinguishing between strains and origin (luminal vs mucosal compartment). As such, there are currently a number of biomarkers in clinical use but no single one can reliably diagnose IBD or sub-classify cases of IBD into UC or CD. The significance of leaving patients without a clear diagnosis is the potential adverse impact on future management. Endoscopy evaluation is currently viewed as the nearest to a ‘gold standard’ tool, however it is often unattractive to patients in terms of comfort and convenience. In addition, colonoscopy may present some significant risk such as perforation. It is estimated that up to 50% of patients with gastrointestinal symptoms are referred for unnecessary endoscopic investigation.

Faecal biomarkers represent an attractive non-invasive alternative indicator of IBD since they are more acceptable to patients and easier to perform in everyday clinical practice. To date, faecal markers include a biologically heterogeneous group of substances that either leak from or are actively released by the inflamed mucosa (such as calprotectin or lactoferrin) but these biomarkers are not specific for IBD or cannot distinguish between UC and CD.

It is therefore an aim of the present invention to identify a microbial gene the presence of which can be utilised to address the abovementioned problems.

It is a further aim of the present invention to provide a method of identifying and/or inhibiting a transporter protein to address the abovementioned problems.

It is a yet further aim of the present invention to provide a microbial-derived faecal biomarker which addresses the abovementioned problems.

In a first aspect of the invention there is provided a method of identifying, monitoring and/or diagnosing mucosal bacterial presence or infection, said method including the step of detecting at least part of a sialic acid transporter protein encoded by Ruminococcus gnavus (R. gnavus) ATCC 29149 Nan cluster.

Typically the transporter protein is specific to 2,7-anhydro-Neu5Ac.

In one embodiment the substrate or solute binding protein of the ATCC 29149 Nan cluster is encoded by RUMGNA_02698.

Typically the transporter protein is used as an indicator or biomarker for inflammatory bowel disease. Further typically the transporter protein is used as a faecal biomarker.

In one embodiment the presence of the transporter protein is used as an indicator of likelihood of success of microbiome-targeted therapies such as faecal microbiota transplantation.

In one embodiment polymerase chain reaction (PCR) is used to amplify the protein and/or identify the presence of the transporter protein. Typically quantitative polymerase chain reaction (qPCR) is used to identify the presence of the protein.

In one embodiment the presence or absence of the transporter protein is used to distinguish or diagnose UC or CD.

In a second aspect of the invention there is a method of inhibition of the growth of bacterium, said method including the step of inhibition of a sialic acid transporter protein.

Typically the bacterium is Ruminococcus gnavus, Blautia obeum or Streptococcus pneumoniae.

Preferably the bacterium is R. gnavus.

Typically the transporter protein is encoded by ATCC 29149 Nan cluster.

Typically the transporter protein is specific to 2,7-anhydro-Neu5Ac.

In one embodiment the substrate or solute binding protein of the ATCC 29149 Nan cluster is encoded by RUMGNA_02698.

In one embodiment the transporter (SBP) is not the only gene-specific to R. gnavus Nan cluster, typically the RgOx is also specific to the peculiar cluster as needed to convert 2,7-anhydro-Neu5Ac to Neu5Ac once inside the cell.

Further typically, a biomarker could be either RgSBP or RgOx, or the whole cluster of genes.

In a third aspect of the invention there is provided a method of treatment of a mucosal disease in a subject comprising administering a therapeutically effective amount of a transport protein inhibitor.

In a further aspect of the invention there is provided a pharmaceutical composition including a transport protein inhibitor.

Typically the transporter protein is specific to 2,7-anhydro-Neu5Ac.

Further typically the inhibition is by direct or indirect inhibition.

The skilled person will appreciate the advantages over current methods is that, without being invasive, the present invention provides an indication of bacterial strains reflecting the aberrant glycosylation (particularly in IBD patients) at the mucosal level. This can be monitored by a targeted qPCR test using stored faecal material. It is also rapid, simple (more practical as compared to biopsies) and low in cost (much cheaper that high throughput sequencing).

Specific embodiments and detail of aspects of the invention are now described with reference to the following figures wherein:

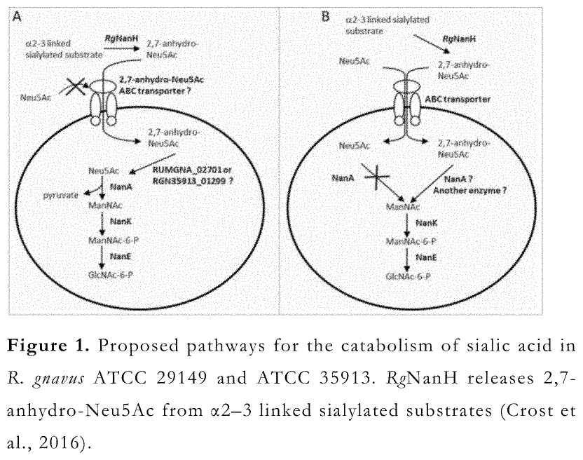

FIG. 1 shows a diagram of proposed pathways for the catabolism of sialic acid in R. gnavus ATCC 29149 and ATCC 35913. RgNanH releases 2,7-anhydro-Neu5Ac from α2-3 linked sialylated substrates;

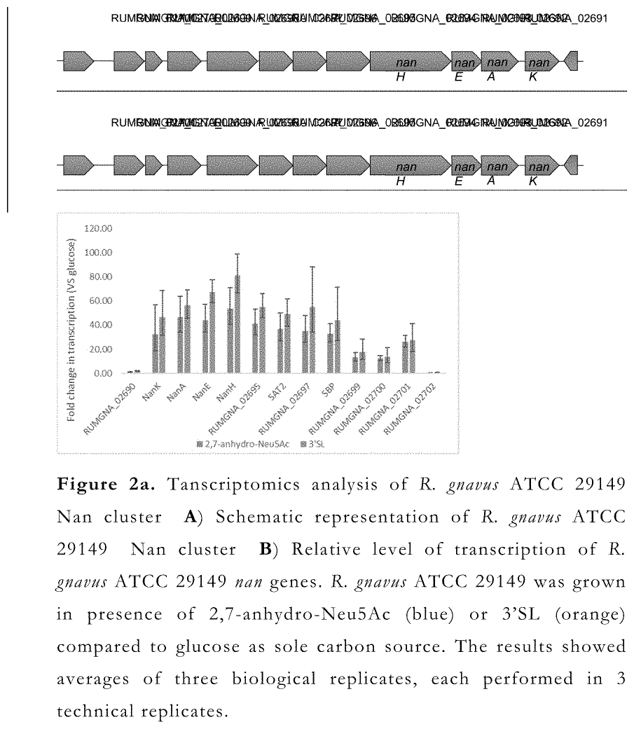

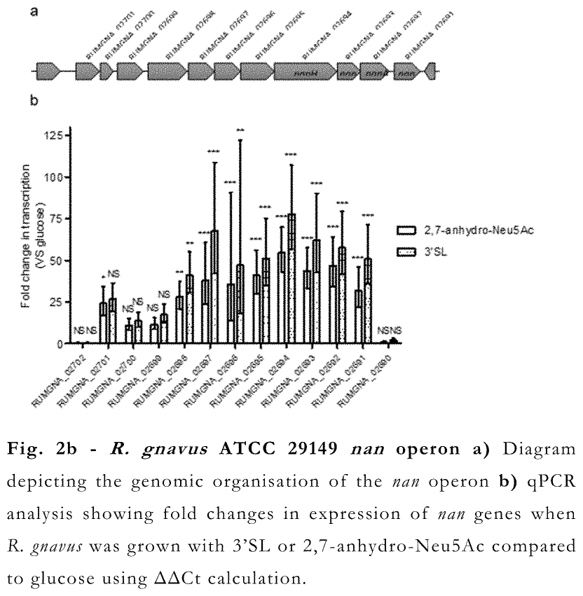

FIG. 2a is a diagram and graph of tanscriptomic analysis of R. gnavus ATCC 29149 Nan cluster;

FIG. 2b shows R. gnavus ATCC 29149 nan operon analysis where 2b (a) is a diagram depicting the genomic organisation of the nan operon, and 2b (b) is a graph of qPCR analysis showing fold changes in expression of nan genes when R. gnavus was grown with 3'SL or 2,7-anhydro-Neu5Ac compared to glucose using ΔΔCt calculation;

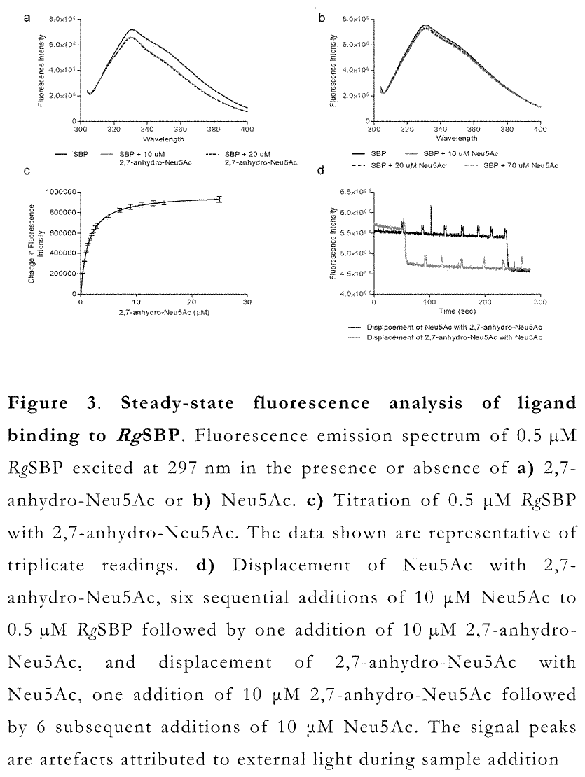

FIG. 3 shows the graphs of fluorescence emission spectrum of steady-state fluorescence analysis of ligand binding to RgSBP. 0.5 μM RgSBP excited at 297 nm in the presence or absence of a) 2,7-anhydro-Neu5Ac or b) Neu5Ac. c) Titration of 0.5 μM RgSBP with 2,7-anhydro-Neu5Ac. The data shown are representative of triplicate readings. d) Displacement of Neu5Ac with 2,7-anhydro-Neu5Ac;

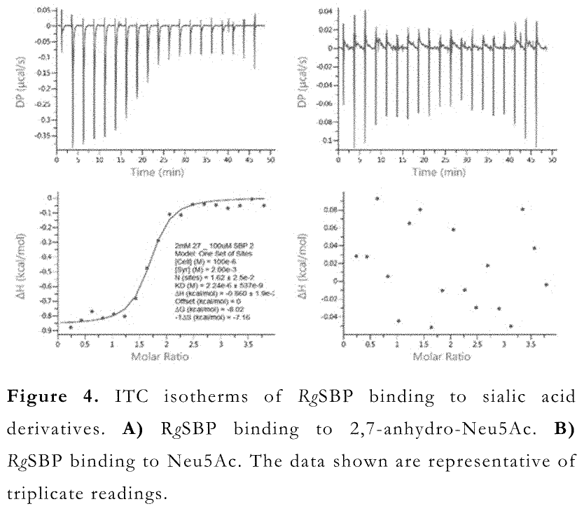

FIG. 4 shows ITC isotherms of RgSBP binding to sialic acid derivatives where A) RgSBP binding to 2,7-anhydro-Neu5Ac and B) RgSBP binding to Neu5Ac;

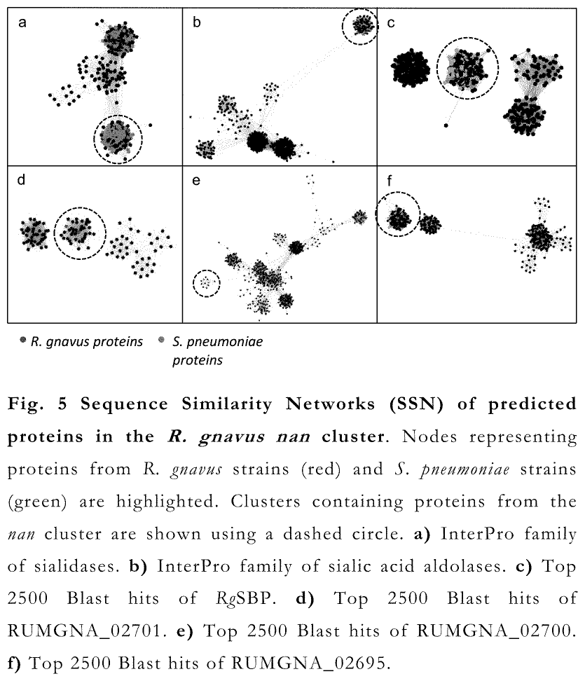

FIG. 5 shows Sequence Similarity Networks (SSN) of predicted proteins in the R. gnavus nan cluster. Nodes representing proteins from R. gnavus strains (red) and S. pneumoniae strains (green) are highlighted. Clusters containing proteins from the nan cluster are shown using a dashed circle, a) InterPro family of sialidases, b) InterPro family of sialic acid aldolases, c) Top 2500 Blast hits of RgSBP, d) Top 2500 Blast hits of RUMGNA_02701, e) Top 2500 Blast hits of RUMGNA_02700, f) Top 2500 Blast hits of RUMGNA_02695;

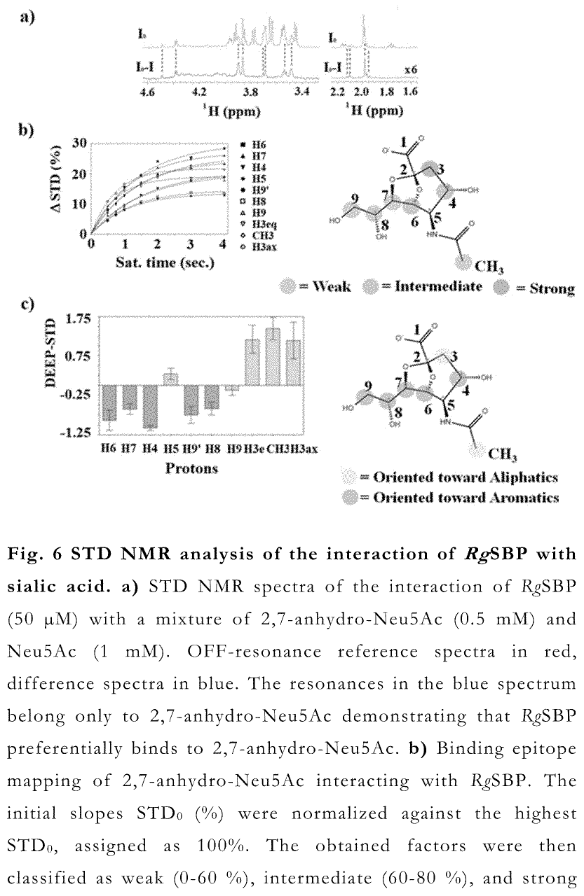

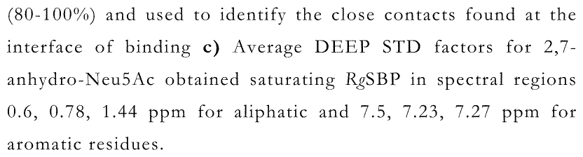

FIG. 6 shows STD NMR analysis of the interaction of RgSBP with sialic acid, where a) STD NMR spectra of the interaction of

RgSBP (50 μM) with a mixture of 2,7-anhydro-Neu5Ac (0.5 mM) and Neu5Ac (1 mM), wih OFF-resonance reference spectra in red, difference spectra in blue. The resonances in the blue spectrum belong only to 2,7-anhydro-Neu5Ac demonstrating that RgSBP preferentially binds to 2,7-anhydro-Neu5Ac. b) Binding epitope mapping of 2,7-anhydro-Neu5Ac interacting with RgSBP. The initial slopes STD₀ (%) were normalized against the highest STD₀, assigned as 100%. The obtained factors were then classified as weak (0-60%), intermediate (60-80%), and strong (80-100%) and used to identify the close contacts found at the interface of binding c) Average DEEP STD factors for 2,7-anhydro-Neu5Ac obtained saturating RgSBP in spectral regions 0.6, 0.78, 1.44 ppm for aliphatic and 7.5, 7.23, 7.27 ppm for aromatic residues;

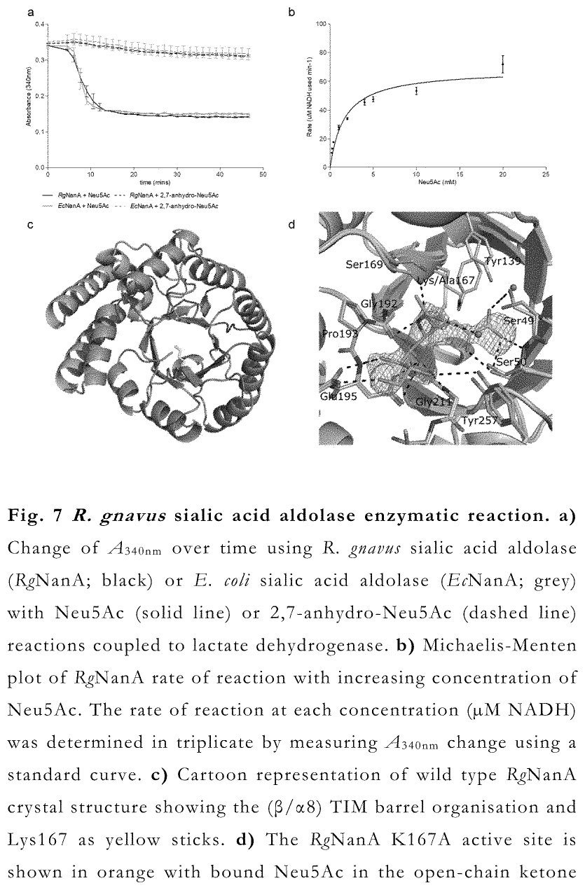

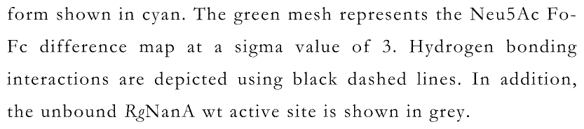

FIG. 7 R. gnavus sialic acid aldolase enzymatic reaction. a) Change of A_{340 nm} over time using R. gnavus sialic acid aldolase (RgNanA; black) or E. coli sialic acid aldolase (EcNanA; grey) with Neu5Ac (solid line) or 2,7-anhydro-Neu5Ac (dashed line) reactions coupled to lactate dehydrogenase. b) Michaelis-Menten plot of RgNanA rate of reaction with increasing concentration of Neu5Ac. The rate of reaction at each concentration (μM NADH) was determined in triplicate by measuring A_{340 nm} change using a standard curve. c) Cartoon representation of wild type RgNanA crystal structure showing the (β/α8) TIM barrel organisation and Lys167 as yellow sticks. d) The RgNanA K167A active site is shown in orange with bound Neu5Ac in the open-chain ketone form shown in cyan. The green mesh represents the Neu5Ac Fo-Fc difference map at a sigma value of 3. Hydrogen bonding interactions are depicted using black dashed lines. In addition, the unbound RgNanA wt active site is shown in grey;

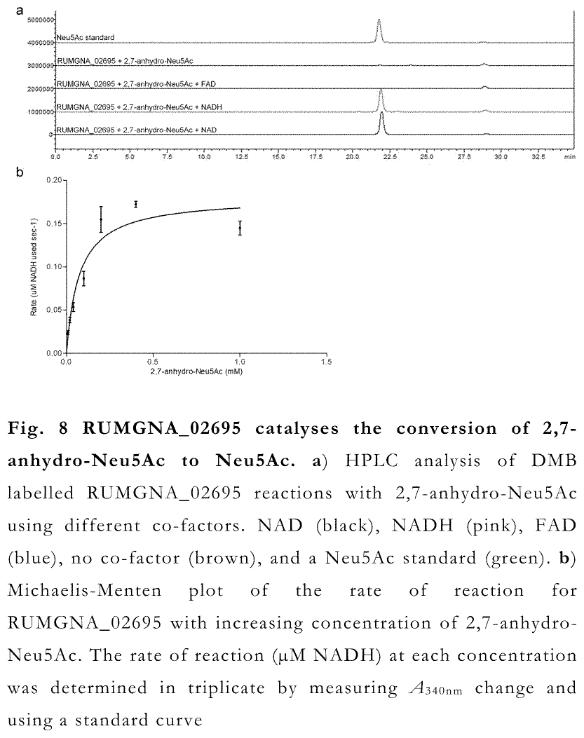

FIG. 8 shows RUMGNA_02695 catalyses the conversion of 2,7-anhydro-Neu5Ac to Neu5Ac, where a) HPLC analysis of DMB labelled RUMGNA_02695 reactions with 2,7-anhydro-Neu5Ac using different co-factors. NAD (black), NADH (pink), FAD (blue), no co-factor (brown), and a Neu5Ac standard (green). b) Michaelis-Menten plot of the rate of reaction for

RUMGNA_02695 with increasing concentration of 2,7-anhydro-Neu5Ac. The rate of reaction (μM NADH) at each concentration was determined in triplicate by measuring A_{340 nm} change and using a standard curve;

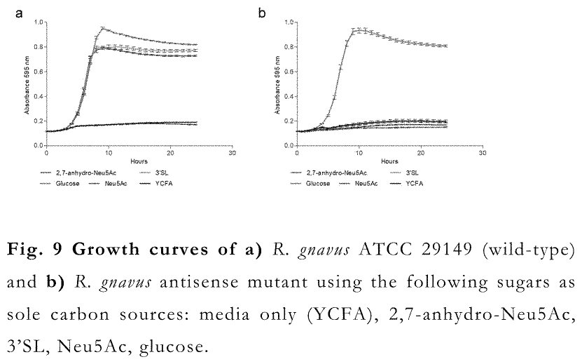

FIG. 9 shows growth curves of a) R. gnavus ATCC 29149 (wild-type) and b) R. gnavus antisense mutant using the following sugars as sole carbon sources: media only (YCFA), 2,7-anhydro-Neu5Ac, 3'SL, Neu5Ac, glucose;

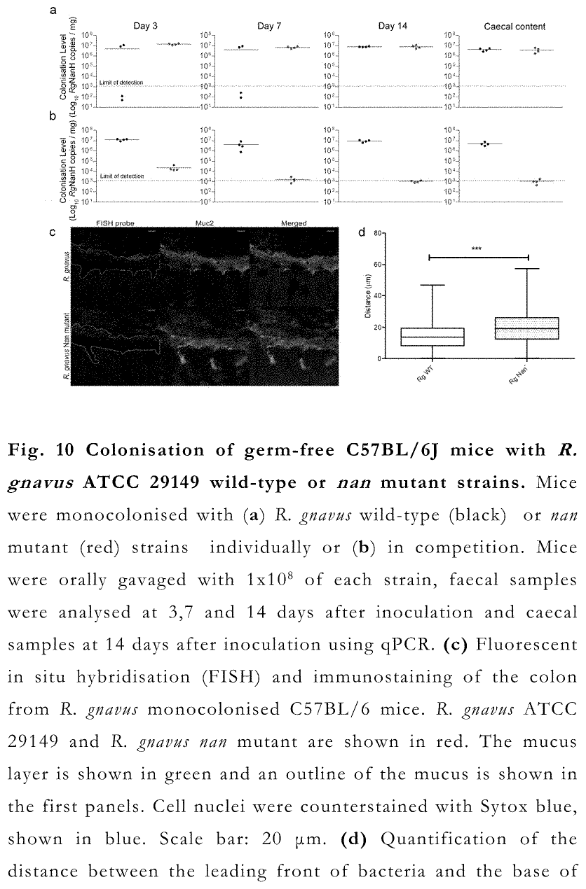

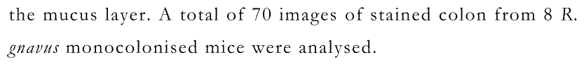

FIG. 10 shows the colonisation of germ-free C57BL/6J mice with R. gnavus ATCC 29149 wild-type or nan mutant strains. Mice were monocolonised with (a) R. gnavus wild-type (black) or nan mutant (red) strains individually or (b) in competition. Mice were orally gavaged with 1×10⁸ of each strain, faecal samples were analysed at 3,7 and 14 days after inoculation and caecal samples at 14 days after inoculation using qPCR. (c) Fluorescent in situ hybridisation (FISH) and immunostaining of the colon from R. gnavus monocolonised C57BL/6 mice. R. gnavus ATCC 29149 and R. gnavus nan mutant are shown in red. The mucus layer is shown in green and an outline of the mucus is shown in the first panels. Cell nuclei were counterstained with Sytox blue, shown in blue. Scale bar: 20 μm. (d) Quantification of the distance between the leading front of bacteria and the base of the mucus layer. A total of 70 images of stained colon from 8 R. gnavus monocolonised mice were analysed;

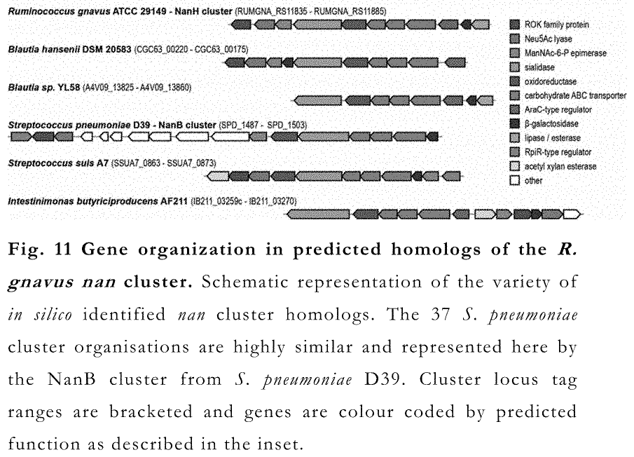

FIG. 11 shows a schematic representation of gene organization in predicted homologs of the R. gnavus nan cluster. In the variety of in silico identified nan cluster homologs, the 37 S. pneumoniae cluster organisations are highly similar and represented here by the NanB cluster from S. pneumoniae D39. Cluster locus tag ranges are bracketed and genes are colour coded by predicted function as described in the inset; and

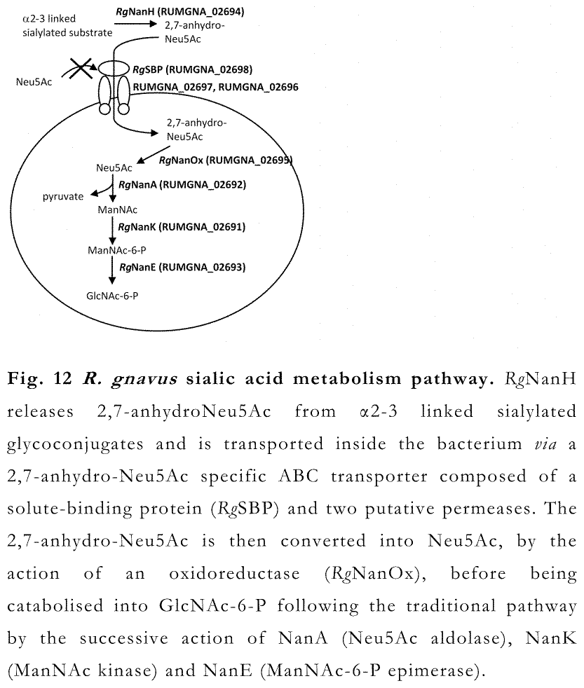

FIG. 12 shows a schematic of the indicated R. gnavus sialic acid metabolism pathway. RgNanH releases 2,7-anhydroNeu5Ac from α2-3 linked sialylated glycoconjugates and is transported inside the bacterium via a 2,7-anhydro-Neu5Ac specific ABC transporter composed of a solute-binding protein (RgSBP) and two putative permeases. The 2,7-anhydro-Neu5Ac is then converted into Neu5Ac, by the action of an oxidoreductase (RgNanOx), before being catabolised into GlcNAc-6-P following the traditional pathway by the successive action of NanA (Neu5Ac aldolase), NanK (ManNAc kinase) and NanE (ManNAc-6-P epimerase).

### EXPERIMENTAL

### 2,7-anhydro-Neu5Ac Induces Expression of the Entire Nan Cluster

Based on the transcriptomics analyses of R. gnavus ATCC 29149 and ATCC 35913 on mucin as reported in Crost et al. 2016, we proposed two models for 2,7-anhydro-Neu5Ac metabolism (FIG. 1). In (A) 2,7-anhydro-Neu5Ac could be transported inside the bacterium via a 2,7-anhydro-Neu5Ac-specific ABC transporter composed of a solute-binding protein (RUMGNA_02698 in ATCC 29149; RGNV35913_01296 in ATCC 35913) and two putative permeases (RUMGNA_02697 and RUMGNA_02696 in ATCC 29149; RGNV35913_01295 and RGNV35913_01294 in ATCC 35913) and then hydrolyzed into Neu5Ac, possibly by the action of RUMGNA_02701 or RGNV35913_01299, before being catabolized into GlcNAc-6-P following the traditional pathway by the successive action of NanA (Neu5Ac lyase), NanK (ManNAc kinase) and NanE (ManNAc-6-P epimerase). Alternatively (B), both 2,7-anhydro-Neu5Ac and Neu5Ac could enter the cells via the ABC transporter but NanA would either be inactive or specific for 2,7-anhydro-Neu5Ac, explaining the absence of growth of the bacteria on sialic acid (see FIG. 1).

To further assess the contribution of the nan genes in the metabolism of 2,7-anhydro-Neu5Ac and taking advantage of our synthetic approach to produce milligram amounts of 2,7-anhydro-Neu5Ac (Monestier et al., 2016), we analyzed the transcriptional activity of this cluster by qRT-PCR in R. gnavus ATCC 29149 grown on 2,7-anhydro-Neu5Ac or α2-3-sialyllactose (3'SL) as sole carbon source. We showed that the expression of all genes constituting the Nan cluster was induced upon bacterial growth on 2,7-anhydro-Neu5Ac or 3'SL as compared to glucose using ΔΔCt calculations whereas the expression of the two genes flanking the cluster (RUMGNA_02702, RUMGNA_02690) remained unchanged (FIG. 2).

The change in transcription of the nan genes was between 20 and 80 fold for 3'SL or 2,7-anhydro-Neu5Ac as compared to glucose and this increase was statistically significant for all 11 genes of the operon (to do). There was no significant difference between the change in expression for growth on 2,7-anhydro-Neu5Ac as compared to 3'SL. These results indicate that in R. gnavus ATCC 29149, the Nan operon is adapted to the metabolism of 2,7-anhydro-Neu5Ac from host sialoglycans.

### Bioinformatics Analysis of R. gnavus Nan Cluster

MultiGeneBlast analysis of the R. gnavus Nan cluster revealed that the cluster dedicated to 2,7-anhydro-Neu5Ac utilisation is shared by a limited number of species including two closely related Blautia strains and Streptococcus pneumoniae. (analysis carried by Jan Claessen with help from Emmanuelle Crost). This finding supports the specialisation of the R. gnavus Nan cluster, conferring the bacteria with a unique advantage over other members of the gut microbiota to colonise the mucus niche in the human colon.

### The Sialic Acid Transporter is Specific for 2,7-anhydro-Neu5Ac

The Nan cluster in R. gnavus ATCC 29149 is predicted to encode a putative sialic acid transporter of the SAT2 family, with RUMGNA_02698 predicted to be a solute binding protein (SBP) and RUMGNA_02697 and 02696 predicted to be two permeases (Crost et al., 2013; 2016). To determine the ligand specificity of R. gnavus SBP protein (RgSBP), the corresponding gene was amplified by PCR from the R. gnavus ATCC 29149 genome, cloned and heterologously expressed in E. coli and the His₆-tag recombinant protein purified by immobilised metal ion affinity chromatography (IMAC).

Ligand binding to RgSBP was investigated by measuring changes in the intrinsic protein fluorescence upon addition of 2,7-anhydro-Neu5Ac or Neu5Ac as potential ligands. (Andrew Bell with help from student in Gavin Thomas's Group,York). Due to the presence of tyrosine residues in RgSBP, fluorescence changes were measured by exciting at 297 nm. Under these conditions the protein has a maximal emission at 331 nm. Addition of 10 μM or 20 μM 2,7-anhydro-Neu5Ac resulted in a change in the spectrum intensity with a significant shift at 350 nm. 2,7-Anhydro-Neu5Ac caused a 16% quench in the fluorescence of the protein at ligand saturation (FIG. 3A). In marked contrast, addition of Neu5Ac at 10 μM, 20 μM or 70 μM did not lead to any change in the spectra, indicating a lack of binding (FIG. 3B).

Together these data clearly show that RgSBP specifically binds to 2,7-anhydro-Neu5Ac but not to Neu5Ac, in line with the reported growth of R. gnavus ATCC 29149 on this substrate (Crost et al., 2016), conferring an adaptive advantage for R. gnavus to colonise the colonic mucus niche.

The binding of RgSBP to 2,7-anhydro-Neu5Ac or Neu5Ac was further investigated by measuring changes in fluorescence emission at 350 nm when RgSBP was excited at 297 nm upon sequential additions of 10 μM ligands. Following six sequential additions of 10 μM Neu5Ac, no change in intensity was observed at 350 nM, whereas the subsequent addition of 10 μM 2,7-anhydro-Neu5Ac led to a large decrease in intensity (FIG. 3C). Conversely, addition of 10 μM 2,7-anhydro-Neu5Ac resulted in a large decrease in intensity and 6 subsequent additions of 10 μM Neu5Ac caused no further reduction in the intensity (FIG. 3D), further supporting the specificity of the interaction between RgSBP and 2,7-anhydro-Neu5Ac.

The affinity of the interaction between RgSBP and sialic acid ligands was further assessed by ITC.

RgSBP bound to 2,7-anhydro-Neu5Ac with a K_{d} of 2.42±0.27 μM (FIG. 4A). No binding was observed when Neu5Ac was used as the ligand (FIG. 4B), in agreement with the findings from fluorescence spectroscopy.

Subsequent studies have shown that The substrate binding protein, which forms part of a novel SAT3 sialic transporter in R. gnavus ATCC29149, is specific to 2,7-anhydro-Neu5Ac, as shown by fluorescence spectroscopy, isothermal titration calorimetry (ITC), and saturation transfer difference nuclear magnetic resonance spectroscopy (STD NMR). Once inside the cell, 2,7-anhydro-Neu5Ac is converted into Neu5Ac via a novel enzymatic reaction catalysed by an oxidoreductase, RgNanOx. Following this conversion, Neu5Ac is then catabolised into ManNAc and pyruvate via the action of a Neu5Ac-specific aldolase that is structurally and biochemically typical of NanA-like enzymes, as shown by X-ray crystallography of RgNanA wild-type and site-directed active site mutant K167A in complex with Neu5Ac. We confirmed the importance of this metabolic pathway in vivo by generating a R. gnavus nan cluster deletion mutant that lost the ability to grow on sialylated substrates. We showed that in gnotobiotic mice colonised with R. gnavus wild-type and mutant strains, the fitness of the nan mutant was significantly impaired as compared to the wild-type strain with a reduced ability to colonise the mucus layer. Overall, our study revealed a novel sialic acid pathway in bacteria, which has significant implications for the spatial adaptation of mucin-foraging gut symbionts in health and disease.

Thus we have that the entire cluster was induced when R. gnavus ATCC 29149 was grown in the presence of 3'SL or 2,7-anhydro-Neu5Ac, indicating that the nan operon is adapted to the metabolism of 2,7-anhydro-Neu5Ac from host sialoglycans. Before being metabolised, a functional sialic acid transporter is essential for the uptake of sialic acid derivatives into the bacterial cell. The R. gnavus ATCC 29149 nan cluster contains a single ABC transporter (RUMGNA_02696-8) which is orthologous to the S. pneumoniae SAT3 system of unknown function (Sp_1690-2). By studying RgSBP subunit, we have discovered that this is a specific transporter for 2,7-anhydro-Neu5Ac with a K_{d} of 2.42±0.27 μM, which does not bind Neu5Ac, therefore providing the first biochemical characterisation of a SAT3 sialic acid transporter. The low affinity as compared to bacterial SatA transporters specific for Neu5Ac characterised to date, which bind in the nM range (Gangi et al., 2018), might be consistent with the ‘exclusive’ access of the bacteria to the 2,7-anhydro-Neu5Ac substrate. As the transporter lacks its ATPase subunit, it is expected to be coupled with an MsiK-like ATPase encoded elsewhere in the R. gnavus genome. RUMGNA_03040 has the highest degree of homology to the Streptococcus pneumoniae MsiK protein with 59% identity. Taken together these findings indicate that the ability of R. gnavus strains to grow on 2,7-anhydro-Neu5Ac (and not on Neu5Ac) can be explained by the exquisite specificity of RgSBP (RUMGNA_02698) which forms part of the SAT2 sialic transporter (with RUMGNA_02697 and RUMGNA_02696 permeases).

Once inside the cell, 2,7-anhydro-Neu5Ac needs to be converted back into Neu5Ac to become a substrate for the sialic acid aldolase. We identified a novel enzymatic reaction catalysed by RgNanOx, an oxidoreductase (RUMGNA_02695). Interestingly the enzyme is able to convert Neu5Ac into 2,7-anhydro-Neu5Ac in the presence of NAD+ or NADH in a reversible manner but with no net change in NADH concentration, suggesting a novel enzymatic reaction which detailed mechanism of action remains to be determined. Bioinformatic analysis identified close homologous of this protein in a range of bacterial species including YjhC from E. coli and B0186_05960 from Haemophilus haemoglobinophilus (FIG. 2). Neu5Ac is then converted into ManNAc and pyruvate via the action of RgNanA (RUMGNA_02692), a Neu5Ac-specific aldolase, as shown by enzymatic assays and confirmed by the crystal structure of the complex between RgNanA inactive mutant and Neu5Ac. Together these data provide robust biochemical evidence for a new sialic metabolism pathway in bacteria.

Further we confirmed the importance of this metabolic pathway by generating a R. gnavus nan deletion mutant that was tested in vitro and in vivo using gnotobiotic mice colonised with R. gnavus wild-type and/or mutant strains. In in vivo competition experiments, the fitness of the mutant was impaired as compared to the wild-type strain with a reduced ability to colonise the mucus layer as demonstrated by FISH staining. The nan cluster is therefore important to maintain the spatial distribution of R. gnavus strains in the gut. The ability for R. gnavus strains harbouring a nan cluster to penetrate further down into the mucus layer as shown here by confocal microscopy may contribute to protect the bacteria from the constant mucus turner-over. This mechanism may serve as a determinant underlying R. gnavus success as one of the most largely shared species among individuals (Qin et al., 2010; Kraal et al., 2014). Together these findings provide first biochemical and in vivo evidence for the role of R. gnavus nan cluster in the adaptation of this important gut symbiont to the mucosal environment in the gut.

### Materials and Methods

### Materials

All chemicals were obtained from Sigma (St Louis, USA) unless otherwise stated. D-glucose (Glc), N-acetylneuraminic acid (Neu5Ac), were purchased from Sigma-Aldrich (St Louis, Mo.). 3'-sialyllactose (3'SL) was purchased from Carbosynth Limited (Campton, UK). 2,7-anhydro-Neu5Ac was prepared as previously described (Monestier et al., 2017; Xiao et al., 2018).

### Bacterial Strains and Media

R. gnavus ATCC 29149 was routinely grown in an anaerobic cabinet (Don Whitley, Shipley, UK) in BHI-YH as previously described (Crost et al., 2013). Growth on single carbon sources utilized anaerobic basal YCFA medium (Duncan et al., 2002) supplemented with 11.1 mM of specific mono- or oligosaccharides (2,7-anhydro-Neu5Ac, 3'Sialyllactose (3'SL) or glucose). The bacteria were grown to late exponential phase for RNA extraction, the culture was performed in 14 ml tubes. Growth was determined spectrophotometrically by monitoring changes in optical density at 600 nm compared to the same medium without bacterium (ΔOD_{595 nm}) hourly for 10 hours.

### Quantitative Real-Time PCR (qRT-PCR)

Total RNA was extracted from 3 ml of mid- to late exponential phase cultures of R. gnavus ATCC 29149 in YCFA supplemented with one carbon source (Glc, 3'SL or 2,7-anhydro-Neu5Ac). Three biological replicates were performed for each carbon source. The RNA was stabilized prior to extraction by using RNAprotect Bacteria Reagent (Qiagen, Crawley, UK) according to the manufacturer's instructions. The RNA was then extracted after an enzymatic lysis followed by a mechanical disruption of the cells, using the RNeasy Mini Kit (Qiagen) according to manufacturer's instructions with an on-column DNAse treatment. The purity and quantity of the extracted RNA was assessed with NanoDrop 1000 UV-Vis Spectrophotometer (Thermo Fischer Scientific, Wilmington, Del.) and with Qubit 2.0 (Invitrogen).

qPCR was carried out in an Applied Biosystems 7500 Real-Time PCR system (Life Technologies Ltd). One pair of primers was designed for each target gene using ProbeFinder version 2.45 (Roche Applied Science, Penzberg, Germany) to obtain an amplicon of around 60-80 bp long. The primers were between 18 and 23 nt-long, with a Tₘ of 59-60° C. (Table S1). Calibration curves were prepared in triplicate for each pair of primers using 2.5-fold serial dilutions of R. gnavus ATCC 29149 genomic DNA.

The standard curves showed a linear relationship of log input DNA vs. the threshold cycle (C_{T}), with acceptable values for the slopes and the regression coefficients (R²). The dissociation curves were also performed to check the specificity of the amplicons. Each DNAse-treated RNA (1 μg) was converted into cDNA using QuantiTect® Reverse Transcription kit (Qiagen) according to the manufacturer's instructions. DNAse-treated RNA was also treated the same way but without addition of the reverse-transcriptase (RT-). Each qPCR reaction (10 μl) was then carried out in triplicate with 1 μl of 1 ng/μl (cDNA or RT-) and 0.2 μM of each primer, using the QuantiFast SYBR Green PCR kit (Qiagen) according to the manufacturer's instructions (except for the combined annealing/extension step which was extended to 35 s). Data obtained with cDNA were analyzed only when C_{T} values above 36 were obtained for the corresponding RT-. For each cDNA sample, the 3 C_{T} values obtained for each gene were analyzed using the 2^{-ΔΔCT} method using housekeeping gyrB (RUMGNA_00867) gene as a reference gene and glucose as a reference condition. For each gene in each condition, the final value of the relative level of transcription (expressed as a fold change in gene transcription compared to glucose) is an average of 3 biological replicates.

Cloning, expression, mutagenesis and purification of recombinant proteins

R. gnavus ATCC 29149 genomic DNA (gDNA) was purified from the cell pellet of a bacterial overnight culture (1 ml) following centrifugation (5,000 g, 5 min) using the GeneJET Genomic

DNA Purification Kit (ThermoFisher, UK), according to the manufacturer's instructions.

The full-length RgSBP excluding the signal sequence (residues 1-29), the full length RgNanA and full length RUMGNA_02695 were amplified from R. gnavus ATCC 29149 gDNA, and cloned into the pHISTEV expression system, introducing a His-tag at the N terminus using primers listed in Table S1. DNA manipulation was carried out in E. coli DH5α cells. Sequences were verified by DNA sequencing by Eurofins MWG (Ebersberg, Germany) following plasmid preparation using the Monarch Plasmid Miniprep kit (New England Biolabs). The RgNanA active site mutant, K167A, was generated using the QuikChange Lightning mutagenesis kit (Agilent) and primers listed in Table S1. E. coli BL21 (New England BioLabs) cells were transformed with the recombinant plasmid harbouring the gene of interest according to manufacturer's instructions. Expression was carried out in 800 ml ‘Terrific Broth Base with Trace Elements’ autoinduction media (ForMedium, Dundee, UK) growing cells for 3 h at 37° C. and then at 16° C. for 48 h, with shaking at 250 rpm. The cells were harvested by centrifugation at 10,000 g for 20 min. The His-tagged proteins were purified by immobilized metal affinity chromatography (IMAC) and further purified by gel filtration (Superdex 75 column) on an Akta system (GE Health Care Life Sciences, Little Chalfont, UK). Protein purification was assessed by standard SDS-polyacrylamide gel electrophoresis using NuPAGE Novex 4-12% Bis-Tris gels (Life Technologies, Paisley, UK). Protein concentration was measured with NanoDrop 1000 UV-Vis

Spectrophotometer (Thermo Fischer Scientific, Wilmington, Del.) and using the extinction coefficient calculated by Protparam (ExPASy-Artimo, 2012) from the peptide sequence.

### Fluorescence Spectroscopy

All protein fluorescence experiments used a FluoroMax 3 fluorescence spectrometer with connecting water bath at 37° C. Because of the presence of 15 tyrosine residues, the protein was excited at 297 nm with slit widths of 5 nm. RgSBP was used at a concentration of 0.2 μM in 50 mM Tris pH 7.5 for all fluorescence experiments. Cumulative fluorescence changes from titration of the protein with ligand were plotted in GraphPad and fitted to a single rectangular hyperbola. The K_{d} values reported were averaged from three separate ligand titration experiments.

### Isothermal Titration Calorimetry (ITC)

Isothermal titration calorimetry (ITC) experiments were performed using the PEAQ-ITC system (Malvern, Malvern, UK) with a cell volume of 200 μl. Prior to titration, protein samples were exhaustively dialysed into 50 mM Tris-HCl pH 7.5. The ligand was dissolved in the dialysis buffer. The cell protein concentration was 100 μM and the syringe ligand concentration was 2 mM. Controls with titrant (sugar) injected into the buffer only were subtracted from the data. The analysis was performed using the Malvern software, using a single-binding site model. Experiments were carried out in triplicate.

### Sialic Acid Aldolase Activity Assays

Aldolase cleavage was measured by monitoring the decrease in absorbance at 340 nm (A_{340 nm}) as NADH is converted to NAD by lactate dehydrogenase in a coupled reaction where pyruvate is released from sialic acid by the aldolase. Reactions were performed in a 100 μl volume with final concentrations of 150 μM NADH (Sigma, St Louis, USA), 0.5 U LDH (Sigma, St Louis, USA), 10 mM sialic acid (Neu5Ac or 2,7-anhydro-Neu5Ac) and 1.5 μg purified RgNanA or EcNanA (E. coli aldolase CAS: 9027-60-5, Carbosynth, UK) in 50 mM Na-phosphate buffer (pH 7.0). The reactions were performed at 37° C. and monitored using FLUOstar OPTIMA (BMG LABTECH). For kinetics experiments, the sialic acid concentration was varied at 20, 10, 5, 4, 2, 1, 0.4, 0.2, 0.1 mM and the initial rate of reaction determined for each concentration in triplicate before analysis was performed by fitting the data to a Michaelis-Menten using Graph Pad Prism (V 5.03).

To monitor the production of ManNAc during the aldolase-catalyzed reactions, 2-AB labelling was carried out on the products from the above reactions. Briefly, 50 ng GlcNAc was added to 10 μl of each sample as an internal reference, before drying using a Concentrator Plus (Eppendorf). 5 μl of labelling reagent was added and incubated at 65° C. for 3 h. The labelling reagent was prepared by dissolving 50 mg 2-aminobenzamide in a solution containing 300 μl acetic acid and 700 μl DMSO, before 60 mg sodium cyanoborohydride is added. Following addition of H₂O to reach 100 μl total volume, the sample was transferred to a HPLC vial and 10 μl loaded onto a HyperClone 3u ODS (C18) 120A 150×4.6 mm 3 μcolumn. Mobile phases of 0.25% n-butylamine, 0.5% phosphoric acid, 0.1% Tetrahydrofurane; 50% methanol; Acetonitrile and H₂O were used at a 0.7 ml/min flow rate.

### Bioinformatics Analyses

Sequence Similarity Networks (SSN) The InterPro families for RgNanH (Glycoside Hydrolase, family 34; IPR001860) and RgNanA (N-acetylneuraminate lyase; IPR005264) were identified using the UniProt database, this family identifier was used to extract protein sequences using Enzyme Function Initiative (EFI) Enzyme Similarity tool (Gerlt et al., 2015). For the other proteins, the families found in the InterPro database were too large to be analysed, so the sequence BLAST tool was used with a maximum of 2500 protein sequences extracted. From this sequence similarity networks were generated and viewed in Cytoscape version 3.6 (Shannon et al., 2003).

Cluster analysis Homologous gene clusters were identified for the R. gnavus ATCC 29149 nan cluster (Crost et al., 2013) using MultiGeneBlast (Medema et al., 2013). The BCT (Bacteria) GenBank subdivision was queried with the sequence spanning locus tags RUMGMA_RS11835-RUMGNA_RS11885 (from scaffold AAYG02000020_1). The data was manually curated, excluding all clusters that do not contain a predicted sialidase or are homologous to the functionally characterized S. pneumoniae NanC cluster (Xu et al., 2011) and the clusters are summarized by organism and predicted gene content in Table S2.

### RUMGNA_02695 Enzymatic Activity Assay

To assay RUMGNA_02695 activity against 2,7-anhydro-Neu5Ac, the purified recombinant protein was incubated in 100 μl reactions at 37° C. overnight with 1 mM 2,7-anhydro-Neu5Ac, 50 mM sodium phosphate buffer pH 7.0 and 500 μM NADH, NAD, FAD or no cofactor. The reactions were dried using a Concentrator Plus (Eppendorf) for 1 h. Samples were then resuspended in 50 μl of water and 50 μl of reaction buffer (1.74 mg of 1,2-Diamino-4,5-methylenedioxybenzene dihydrochloride (Carbosynth, UK), 324.6 μl MilliQ water, 88.6 μl glacial acetic acid, 58.2 μl of β-Mercaptoethanol and 79.3 μl of sodium hydrosulphite) and incubated for 2 h at 55° C. in the dark. The samples were then centrifuged for 1 min and filtered using a 0.45 μm filter into a glass HPLC vial and directly analysed by HPLC.

DMB-labelled samples were analysed by injecting 10 μl onto a Luna 5 μm C-18(2) LC column 250×4.6 mm (Phenomenex) at 1 ml/min. Mobile phases methanol/acetonitrile/water were used for separation of fluorescently labelled sialic acids. The settings of the fluorescence detector were 373 nm excitation and 448 nm emission. Samples were run alongside a Neu5Ac standard.

To determine the kinetic parameters of RUMGNA_02695 enzymatic reaction, a coupled reaction with lactate dehydrogenase and sialic acid aldolase was carried out as described above but with 15 μg of RgNanA and 10 μg RUMGNA_02695 in each reaction. For the kinetics assays, 1, 0.4, 0.2, 0.1, 0.04, 0.02 and 0.01 mM 2,7-anhydro-Neu5Ac was used and the initial rate of reaction determined for each concentration in triplicate before analysis was performed by fitting the data to a Michaelis-Menten using Graph Pad Prism (V 5.03).

Electrospray ionisation spray mass spectrometry (ESI-MS) analysis was performed using the Applied Biosystems 4000 Q-TRAP. The full 100 μl reaction was diluted with 500 ul of 50% Acetonitrile and 0.1% formic acid and samples analysed in negative ion mode using direct injection.

### ClosTron Mutagenesis

R. gnavus mutants were generated using the ClosTron methodology (Heap et al., 2010), which inserts an erythromycin resistance cassette into the gene of interest. Target sites were identified using the Pertuka method (Perutka et al., 2004). The re-targeted introns were synthesised and ligated into the pMTL007C-E2 vector by ATUM (MenloPark, USA). The plasmids were then transformed into E. coli CA434 using the heat-shock protocol, and the recombinant clones selected for chloramphenicol resistance. Recombinant E. coli cells were grown overnight in 10 ml LB, 1 ml of the overnight culture was pelleted and washed with PBS. The E. coli cell pellet was resuspended in 200 μl of an R. gnavus overnight culture and the cell suspension spotted onto a non-selective BHI-YH plate. Following incubation for 8 h at 37° C. the bacteria were washed from the plate using PBS and plated onto BHI-YH supplemented with cycloserine (250 μg/ml) and thiamphenicol (15 μg/ml) and grown for 72 h to select against E. coli and for transfer of the plasmid to R. gnavus. Individual colonies were grown in non-selective BHI-YH broth overnight to allow expression of the plasmid and genomic recombination. The culture was then plated onto a BHI-YH medium containing cycloserine (250 μg/ml) and erythromycin (10 μg/ml) to select clones with successful genomic recombination. PCR and sequencing were used to confirm recombination in the gene of interest.

Expression of the nan cluster genes in the generated mutants was assessed as described above using RNA samples from growth on YCFA supplemented with glucose.

The ability of the mutants to utilise sialic acids and sialoconjugates was assessed by supplementing YCFA with 11.1 mM of 2,7-anhydro-Neu5Ac, 3'SL, glucose or Neu5Ac in triplicate 200 μl cultures in 96-well microtiter plates. The OD_{595 nm} was measured hourly for 10 h in an infinite F50 plate reader (Tecan, UK) housed within an anaerobic cabinet connected to Magellan V7.0 software.

### Saturation Transfer Difference (STD) NMR Spectroscopy

An amicon centrifuge filter unit with a 10 kDa MW cut-off was used to exchange the protein in 25 mM d₁₉-2,2-bis (hydroxymethyl)-2,2′,2″-nitrilotriethanol pH* 7.4 (uncorrected for the deuterium isotope effect on the pH glass electrode) D₂O buffer and 50 mM NaCl. 2,7-anhydro-Neu5Ac and Neu5Ac were dissolved in 25 mM d19-2,2-bis(hydroxymethyl)-2,2′,2″-nitrilotriethanol pH* 7.4, 50 mM NaCl. Characterization of ligand binding by Saturation Transfer Difference NMR Spectroscopy (Mayer, M. and Meyer, B. (1999) was performed on a Bruker Avance 800.23 MHz at 298 K. The on- and off-resonance spectra were acquired using a train of 50 ms Gaussian selective saturation pulses using a variable saturation time from 0.5 s to 4 s, for binding epitope mapping determination while only 0.5 s of saturation time for each selected frequency was used to perform the DEEP-STD NMR experiments (Monaco et al., 2017). The water signal was suppressed by using the excitation sculpting technique (Hwang et al., 1995), while the remaining protein resonances were filtered using a T₂ filter of 40 ms. All the spectra were performed with a spectral width of 10 KHz and 32768 data points using 256 or 512 scans. This time due to the absence of a 3D structure it was impossible to derive the resonances for saturation of aliphatic and aromatic residues found in the binding site as required by the DEEP-STD NMR technique. Moreover, being SBP a high molecular weight protein the NMR spectra assignment is precluded. For this we adopted a search for druggable sites strategy using 4-hydroxy-1-oxyl-2,2,6,6-tetramethylpiperidine (TEMPOL) as previously described (Nepravishta et al., 2019). 1H-1H TOCSY spectra of the protein (500 μM) were acquired in the presence and in the absence of TEMPOL (2.5 mM and 12.5 mM). The spectra were performed with a spectral width of 10 kHz using a time domain of 2056 data points in the direct dimension and 32 scans. The indirect dimension was acquired using the non-uniform sampling (NUS) technique acquiring a NUS amount of 50% of the original 256 increments resulting in 64 hypercomplex points. The spectra were processed with the Topspin 3.1 compressed sensing (cs) routine. The final selected resonances were those identified by the TEMPOL PRE effect, and not overlapping with ligand signals. The DEEP-STD NMR data obtained were used to derive the average orientation of the ligand bound to SBP by averaging the DEEP-STD factors obtained from each saturated region. The DEEP-STD NMR and binding epitope mapping analysis were performed using previously published procedures. (Nepravishta et al., 2019; Monaco, Set al., 2017; Mayer and James TL, 2004).

### Crystal Structure Determination

Sitting drop vapour diffusion crystallisation experiments of RgNanA wild-type were set up at a concentration of 20 mg/ml and monitored using the VMXi beamline at Diamond Light Source (Sanchez-Weatherby et al., 2019). The described RgNanA wild-type crystal structure was acquired from a crystal grown in the Morpheus screen (Molecular Dimensions), 0.2 M 1,6-hexandiol, 0.2 M 1-butanol, 0.2 M 1,2-propanediol, 0.2 M 2-propanol, 0.2 M 1,4-butanediol, 0.2 M 1,3-propanediol, 0.1 M Hepes/MOPS pH 6.5, 20% ethylene glycol, 10% PEG 8000. The diffraction experiment was performed at the i24 beamline at Diamond Light Source Ltd at 100K using a of 0.96863 Å. The data were processed with Xia2 making use of aimless, dials, and pointless. The structure was phased using MrBump through CCP4 online and Molrep (Keegan and Winn, 2008; Vagin and Teplvakov, 2010; Krissinel et al., 2018). The protein phases successfully using CdNaI from Clostridium difficile (PDB 4woq) prepared using Chainsaw. Refinement was carried out using Refmac, Buster, and PDB redo (Winn et al., 2003; Langer et al., 2008; Smart et al., 2012; Emsley, 2017; van Beusekom et al., 2018). Coot and ArpWarp was used for model building. Molprobity was used for structure validation (Williams et al., 2018). Due to data anisotropy, initial phasing and model building also made use of data processed using the Autoproc pipeline and STARANISO (Kabsch, 2010; Vonrhein et al., 2011), which additionally uses XDS. It was not possible to crystallise RgNanA wild-type in the presence of Neu5Ac as it caused protein precipitation and Neu5Ac soaking experiments dissolved the crystals. Experiments with RgNanA K167A mutant were set up at a concentration of 25 mg/ml. Diffracting crystals grew in 0.1 M Tris/BICINE pH 8.5, 25% PEG, 20% ethylene glycol, 100 mM MgCl₂, 10% PEG 8000 and diffraction experiments were performed at the i04 beamline at Diamond Light Source using a wavelength of 0.9795Å. The crystal structure was phased with PHASER using RgNanA wild-type crystal structure (McCoy et al., 2018). The crystal was soaked with 5 mM Neu5Ac for 60 sec prior to freezing.

### In Vivo Colonisation and Analyses

The impact of the nan deletion mutation on R. gnavus fitness was assessed by its ability to colonise germ-free C57BL/6J mice. A group of four 7-9 week old germ-free mice were gavaged with 1×10⁸ CFU of R. gnavus ATCC 29149 wild-type or antisense nan mutant in 100 μl PBS, individually or in combination. Faecal samples were collected from each mouse at 3,7 and 14 days post gavage, and caecal content taken at day 14. DNA was extracted from these samples using the MP Biomedicals Fast DNA™ SPIN kit for Soil DNA extraction with the following modifications. The samples were resuspended in 978 μl of sodium phosphate buffer before being incubated at 4° C. for one hour following addition of 122 μl MT Buffer. The samples were then transferred to the lysing tubes and homogenised in a FastPrep® Instrument (MP Biomedicals) 3 times for 40 s at a speed setting of 6.0 with 5 min on ice between each bead-beating step. The protocol was then followed as recommended by the supplier.

Colonisation was quantified using qPCR carried out in an Applied Biosystems 7500 Real-Time PCR system (Life Technologies Ltd). One pair of primers was designed to specifically target R. gnavus wild-type strain by spanning the area of insertion into the nan cluster and one pair of primers was designed to specifically amplify the inserted DNA, therefore targeting the nan mutant (Table S1). The primers were between 18 and 23 nt-long, with a Tₘ of 59-60° C. Standard curves were prepared in triplicate for both primer pairs using a 10-fold serial dilution of DNA corresponding to 1×10⁷ copies of RgNanH/2 ul to 1×10² copies/2 ul diluted in 5 μg/ml Herring sperm DNA. The standard curves showed a linear relationship of log input DNA vs. the threshold cycle (C_{T}), with acceptable values for the slopes and the regression coefficients (R²). The dissociation curves were also performed to check the specificity of the amplicons. Each qPCR reaction (10 μl) was then carried out in triplicate with 2 μl of 1 ng/μl DNA (diluted in 5 μg/ml Herring sperm DNA) and 0.2 μM of each primer, using the QuantiFast SYBR Green PCR kit (Qiagen) according to the manufacturer's instructions (except that the combined annealing/extension step was extended to 35 s instead of 30 s). Data obtained were analysed using the prepared standard curves.

### RNAseq Analysis

For RNAseq analysis, the colonic tissues from mono-colonised mice were gently washed and stored in RNAlater at −80° C. until extraction. RNA extraction was performed using the RNeasy mini kit (QIAGEN) following the manufacturer's instructions for purification of total RNA from animal tissues, including the on-column DNase digestion. Homogenisation was achieved with acid washed glass beads using the FastPrep®-24 (MP Biomedicals, Solon, USA) by 3 intermittent runs of 30 s at 6 m/s speed every 5 min, at room temperature. Elution was performed as recommended with 50 μl RNAse-free water. The quality and concentration of the RNA samples was assessed using NanoDrop 2000 Spectrophotometer Nanodrop, the Qubit RNA HS assay on Qubit® 2.0 fluorometer (Life Technologies) and Agilent RNA 600 Nano kit on Agilent 2100 Bioanalyzer (Agilent Technologies, Stockport, UK).

RNAseq was carried out by Novogene (HK) (Hong Kong). Briefly, mRNA was enriched using oligo(dT) beads, fragmented randomly in fragmentation buffer, followed by cDNA synthesis using random hexamers and reverse transcriptase. After first-strand synthesis, a custom second-strand synthesis buffer (Illumina) was added with dNTPs, RNase H and Escherichia coli polymerase I to generate the second strand by nick-translation. The final cDNA library was obtained after a round of purification, terminal repair, A-tailing, ligation of sequencing adapters, size selection and PCR enrichment. Library concentration was first quantified using a Qubit® 2.0 fluorometer (Life Technologies), and then diluted to 1 ng/μl before checking insert size on an Agilent 2100 and quantifying to greater accuracy by qPCR (library activity >2 nM). Sequencing of the library was carried out on Illumina Hiseq platform and 125/150 bp paired-end reads were generated.

For analysis, the Illumina original raw data were first transformed to Sequenced Reads by base calling and recorded in a FASTQ file, which contains sequence information (reads) and corresponding sequencing quality information. The raw reads were then filtered to remove reads containing adapters or reads of low quality. The mapping to the mouse reference genome was done using TopHat2 (Kim et al, 2013). The mismatch parameter was set to two, and other parameters were set to default.

Appropriate parameters were also set, such as the longest intron length. Filtered reads were used to analyze the mapping status of RNA-seq data to the reference genome. The HTSeq software was used to analyze the gene expression levels, using the union mode (Anders, 2010). In order for the gene expression levels estimated from different genes and experiments to be comparable, the FPKM (Fragments Per Kilobase of transcript sequence per Millions base pairs sequenced) was used to take into account the effects of both sequencing depth and gene length. The differential gene expression analysis was carried out using the DESeq package (Anders and Huber, 2010) and the readcounts from gene expression level analysis as input data. An adjusted p value (padj) cut-off of 0.05 was used to determine differential expressed transcripts.

### Fluorescent In Situ Hybridization (FISH) Staining

For FISH analysis, the colonic tissue was fixed in methacarn (60% dry methanol, 30% chloroform and 10% acetic acid), processed and embedded in paraffin as previously described (Johansson et al., 2011). Tissue sections were prepared at 8-10 μM. Paraffin sections were dewaxed and washed in 95% ethanol. The tissue sections were incubated with 100 μl of Alexa Fluor 555-conjugated Erec482 probe (5′-GCTTCTTAGTCARGTACCG-3′) at a concentration of 10 ng/μl, in hybridisation buffer (20 mM Tris-HCl, pH 7.4, 0.9M NaCl, 0.1% SDS) at 50° C. overnight. The sections were then incubated in a 50° C. prewarmed wash buffer (20m M Tris-HCl, pH 7.4, 0.9 M NaCl) for 20 min. All subsequent steps were performed at 4° C. The sections were washed with PBS, the blocked with TNB buffer (0.5% w/v blocking reagent in 100 mM Tris-HCl, pH 7.5, 150 mM NaCl) supplemented with 5% goat serum. To detect mucin, the sections were then counterstained with a Muc2 antibody (sc-15334) at 1:100 dilution in TNB buffer overnight. The sections were washed in PBS, then goat anti-rabbit antibodies (diluted 1:500) were used for immunodetection. The sections were counterstained with Sytox blue (S11348, ThermoFisher) diluted 1:1000 in PBS and mounted in Prolong gold anti-fade mounting medium. The slides were imaged using a Leica TCS SP2 confocal microscope with a x63 objective. The distance between the leading front of bacteria and the base of the mucus layer was measured with FIJI. A total of 70 images from 8 mice were analysed. The association between genotype and distances was estimated by a linear mixed model, including fixed effects of genotype and area and random effects of mouse and each individual image. There was substantial spatial correlation between adjacent observations and so an AR(1) correlation structure was added. The resulting model had no residual autocorrelation as judged by visual inspection of autocorrelation function. The nmle package version 3.1-137 using R version 3.5.3 was used to estimate the model.

### Results

### 2,7-anhydro-Neu5Ac Induces Expression of the Entire Nan Cluster

The gene encoding the intramolecular trans-sialidase (IT-sialidase; RgNanH) is part of a complete nan cluster (nanAKE) (Crost et al., 2016). In R. gnavus ATCC 29149 , RUMGNA_02699 is a predicted transcriptional regulator, RUMGNA_02698-02696 encode a putative sialic acid ABC transporter of the SAT3 family, RUMGNA_02694 encodes RgNanH and RUMGNA_02693-02691 encode predicted homologs of the canonical nan cluster, RgNanA (aldolase), NanE (epimerase) and NanK (kinase). For the remaining 3 genes RUMGNA_02701 shares homology with sialic acid esterase proteins, 02700 has homology to the YhcH protein family and 02695 is a putative oxidoreductase (Crost et al., 2016). To determine the contribution of the nan genes in the metabolism of 2,7-anhydro-Neu5Ac, we first analysed the transcriptional activity of this cluster by qRT-PCR in R. gnavus ATCC 29149 grown on 2,7-anhydro-Neu5Ac or α2-3-sialyllactose (3'SL) as sole carbon source. We showed that the expression of all genes constituting the nan cluster was induced upon bacterial growth on 2,7-anhydro-Neu5Ac or 3'SL as compared to glucose using ΔΔCt calculations whereas the expression of the two genes flanking the cluster (RUMGNA_02702, RUMGNA_02690) remained unchanged (FIG. 2b).

The change in transcription of the nan genes was between 20- and 80-fold for 3'SL or 2,7-anhydro-Neu5Ac as compared to glucose and this increase was statistically significant for 8 and 9 of the 11 genes of the operon for growth on 3'SL or 2,7-anhydro-Neu5Ac respectively. There was no significant difference between the change in expression for growth on 2,7-anhydro-Neu5Ac as compared to 3'SL. These results indicate that in R. gnavus ATCC 29149 , the nan operon is adapted to the metabolism of 2,7-anhydro-Neu5Ac from host sialoglycans.

### Bioinformatic Analysis of the Nan Cluster Genes

A sequence similarity network (SSN) analysis was conducted to identify the proteins encoded by the nan cluster, which are associated with the ability of the bacteria to metabolise 2,7-anhydro-Neu5Ac over Neu5Ac. As expected, the IT-sialidase from R. gnavus strains (in red) clustered together with proteins from Streptococcus pneumoniae strains (in green) whose genomes are known to encode IT-sialidases (in addition to other sialidases) (Xu et al., 2008; 2011) (FIG. 2a). Other co-occurring bacterial species include Rumminococcus torques, Lactobacillus salivarius, Staphylococcus pseudintermedius, Streptococcus infantis and Streptococcus mitis. Bacterial species clustering for RgNanH, also shared clusters for proteins encoding RUMGNA_02698 (RgSBP), the predicted soluble binding protein giving specificity to ABC transporters, RgNanA (RUMGNA_02692), the first protein of the canonical Neu5Ac metabolism, and RUMGNA_02695, suggesting that these proteins may be associated with 2,7-anhydro-Neu5Ac metabolism (FIG. 5). In contrast, the RUMGNA_02701 and 02700 predicted proteins did not cluster with proteins from the same set of bacteria.

### The Sialic Acid Transporter is Specific for 2,7-anhydro-Neu5Ac

To determine the specificity of the predicted transporter, the gene encoding the predicted SBP was amplified by PCR from the R. gnavus ATCC 29149 genome, cloned into the pHISTEV expression vector, heterologously expressed in E. coli and the His₆-tag recombinant protein purified by immobilised metal ion affinity chromatography (IMAC). Ligand binding to RgSBP was investigated by measuring changes in the intrinsic protein fluorescence upon addition of 2,7-anhydro-Neu5Ac or Neu5Ac as potential ligands. Due to the presence of 7 tryptophan residues in RgSBP, fluorescence changes were measured by exciting at 297 nm. Under these conditions the protein has a maximal emission at 331 nm. Addition of 10 μM or 20 μM 2,7-anhydro-Neu5Ac resulted in a change in the spectrum intensity with a significant shift at 350 nm, with 2,7-anhydro-Neu5Ac causing an ˜16% quench in the fluorescence (FIG. 3a). In marked contrast, addition of Neu5Ac at 10 μM, 20 μM or 70 μM did not lead to any change in the spectra, suggesting a lack of binding (FIG. 3b). Titration of 0.5 μM RgSBP with 2,7-anhydro-Neu5Ac was performed in triplicate and when fit with a hyperbolic curve gave a Kd of 1.349 μM (+/−0.046) (FIG. 3c). To confirm the novel specificity of 2,7-anhydro-Neu5Ac over Neu5Ac we followed sequential changes in fluorescence at 350 nm following additions of 10 μM of the two ligands. When Neu5Ac is added first no change in fluorescence is observed and a quench is observed with the first addition of 2,7-anhydro-Neu5Ac (FIG. 3d). Conversely, when 2,7-anhydro-Neu5Ac is added first the quench is observed and additions of 10 μM Neu5Ac caused no further reduction or reverse in the intensity (FIG. 3d), indicating Neu5Ac is unable to displace 2,7-anhydro-Neu5Ac, further supporting the specificity of the interaction between RgSBP and 2,7-anhydro-Neu5Ac.

The affinity of the interaction between RgSBP and sialic acid ligands was further assessed by isothermal titration calorimetry (ITC).

RgSBP bound to 2,7-anhydro-Neu5Ac with a K_{d} of 2.42±0.27 μM (FIG. 4a) and no binding was observed when Neu5Ac was used as the ligand (FIG. 4b), in agreement with the findings from fluorescence spectroscopy. The binding of 2,7-anhydro-Neu5Ac revealed a thermodynamic signature with both entropic (−TΔS−7.05±0.08 kcal mol⁻¹) and enthalpic (ΔH−0.93±0.03 kcal mol⁻¹) components contributing favourably to the binding process (ΔG−7.99±0.05 kcal mol⁻¹ FIG. 4a).

Together these data clearly showed that RgSBP specifically binds to 2,7-anhydro-Neu5Ac but not to Neu5Ac, in line with the growth profile of R. gnavus ATCC 29149 on these substrates (Crost et al., 2016).

### Molecular Basis for RgSBP Specificity to 2,7-anhydro-Neu5Ac

To gain structural insights into the unique ligand specificity of RgSBP, saturation transfer difference nuclear magnetic resonance spectroscopy (STD NMR) studies were conducted with RgSBP in the presence of 2,7-anhydro-Neu5Ac or Neu5Ac.

The transfer of magnetization as saturation from the protein to the ligand was clearly observed for 2,7-anhydro-Neu5Ac. On the other hand, the complete absence of saturation transfer to Neu5Ac confirmed that this compound is not a binder and that the protein preferentially selects 2,7-anhydro-Neu5Ac (FIG. 6a).

STD NMR epitope mapping and DEEP-STD NMR were used to further characterize the binding and orientation of 2,7-anhydro-Neu5Ac with RgSBP and gain structural insights into the binding pocket. As shown in FIG. 6b, protons H3, H4 and H6 showed the highest STD (%) factors, indicating that these protons make close contacts with the protein and should be found in the interface of binding. On the other hand, protons H7, H8, H9 and protons belonging to the CH₃ group showed lower STD (%) and are therefore expected to be more exposed to the solvent. For the DEEP-STD experiment, in the absence of an available 3D structure for RgSBP, and due to the high molecular weight of RgSBP precluding any possibility of deriving the frequencies of the residues in the binding site, TEMPOL was used as an alternative approach to investigate the putative binding sites of RgSBP. Briefly, following our recent approach (Nepravishta et al., 2019), the broadening of RgSBP signals beyond detection for the resonances affected by TEMPOL in the ¹H-¹H TOCSY spectra, allowed us to identify frequencies corresponding to protein residues in a putative binding area. In this way, for the saturation frequencies to be used in the DEEP-STD NMR experiments we identified the following NMR resonances: 0.6, 0.78, 1.44 ppm for the aliphatic region and 7.5, 7.27, 7.23, 7.14 and 7.0 ppm for the aromatic region. By averaging all the saturated frequencies in the DEEP STD NMR experiments, it was possible to derive the orientation of the ligand with regard to the distinct saturated protein areas in the putative binding site as shown in FIG. 6c. Using this approach, we found that protons H4, H6, H7, H8, H9′ are preferentially oriented toward aromatic residues while H3 and protons belonging to the CH₃ group are oriented toward aliphatic residues.

Taken together the STD NMR data confirmed that RgSBP preferentially binds to 2,7-anhydro-Neu5Ac over Neu5Ac. In addition, using the DEEP-STD NMR, we have been able to characterize the orientation of the ligand in the binding site. The data also confirmed the contribution of aromatic residues such as Trp, Tyr, Phe in the binding site as supported by the fluorescence spectroscopy experiments (above). Moreover, the findings from the DEEP-STD NMR and TEMPOL experiments clearly indicate the presence of aliphatic and aromatic residues in the binding site of RgSBP and that these residues are involved in the binding of 2,7-anhydro-Neu5Ac.

### R. gnavus Sialic Acid Aldolase is Specific for Neu5Ac

The first step of sialic acid metabolism is the conversion of sialic acid to ManNAc and pyruvate catalysed by a sialic acid aldolase (NanA). To determine the substrate specificity of RUMGNA_02692 (RgNanA), the corresponding gene was amplified by PCR from the R. gnavus ATCC 29149 genome, cloned into the pHISTEV expression vector, heterologously expressed in E. coli and the His₆-tag recombinant protein purified by immobilised metal ion affinity chromatography (IMAC). The substrate specificity of RgNanA was determined using a coupled activity assay where pyruvate released during the conversion of sialic acid to ManNAc is converted to lactate by a lactate dehydrogenase and the subsequent loss of absorbance at 340 nm measured as NADH is converted to NAD⁺. A commercially available E. coli sialic acid aldolase (EcNanA) was included as a control and enzymes were tested for activity against 2,7-anhydro-Neu5Ac and Neu5Ac. Both enzymes showed activity against Neu5Ac whilst neither enzyme showed activity against 2,7-anhydro-Neu5Ac (FIG. 7a). The products of these reactions were analysed by HPLC and confirmed that reactions of both enzymes with Neu5Ac produced ManNAc, whereas no reaction product was detected when 2,7-anhydro-Neu5Ac was used as a substrate. The kinetic parameters of RgNanA were determined by calculating the initial rate of reaction with increasing Neu5Ac concentrations. A Michaelis-Menten curve was fitted to the data and kinetic parameters determined (FIG. 7b). The k_{cat} was calculated at 2.757±0.033 s⁻¹ and the K_{M} 1.473±0.098 mM. These values are consistent with other reported data of sialic acid aldolases in bacteria.

The crystal structure of RgNanA wt presents as a (β/α8) TIM barrel with an adjacent three-helix bundle, a fold shared with other bacterial Neu5Ac lyases (Barbosa et al., 2000; Huynh et al., 2013; Timms et al., 2013; North et al., 2016; Campeotto et al., 2018; Kumar et al., 2018). Structural inspection of the RgNanA active site indicates a high degree of similarity with previously characterised sialic acid aldolases (FIG. 7c), supporting RgNanA substrate specificity for Neu5Ac. The RgNanA wt crystals dissolved in Neu5Ac soaking experiments, as also observed previously with Pasteurella multocida Neu5Ac aldolase (Huynh et al., 2013) (PcNanA), which may be due to subtle conformational changes during substrate binding or catalysis. However, following soaking of RgNanA K167A crystals with Neu5Ac, clear electron density for Neu5Ac in the open-chain ketone form was present. Neu5Ac was shown to form extensive interactions with the enzyme active site, with hydrogen bonds to the side chains of Ser49, Ser50, Ser169, Asp194, Glu195, and Tyr257, and main chain atoms of Ser50, Gly192, Asp194, Gly211. The N-acetyl group is oriented out of RgNanA active site. In the active site of the E. coli Neu5Ac lyase/aldolase (EcNanA), Ser47, Tyr110, Tyr137, and Thr167 were identified to be important for catalytic activity (Daniels et al., 2014). These residues are conserved in RgNanA with the exception of E. coli Thr167, which is Ser169 in RgNanA. The EcNanA Thr167 and RgNanA Ser169 hydroxyls superimpose. Notably, the EcNanA T167S mutation did not affect the enzyme kinetic parameters (ref). Comparing the active sites of the wild type and mutant RgNanA protein highlights a 1.8 Å shift by the Tyr139 α-carbon. This movement is also present in the apo crystal structure, therefore presumably due to the absence of Lys167 rather than the presence of Neu5Ac.

### RUMGNA_02695 Catalyses the Conversion of 2,7-anhydro-Neu5Ac to Neu5Ac

To identify the substrate of RUMGNA_02695, the corresponding gene was amplified by PCR from the R. gnavus ATCC 29149 genome, cloned into the pHISTEV expression vector, heterologously expressed in E. coli and the His₆-tag recombinant protein purified by immobilised metal ion affinity chromatography (IMAC). The protein is predicted to include a Rossman fold, so the recombinant protein was incubated with 2,7-anhydro-Neu5Ac in the presence and absence of NAD⁺/NADH/FAD as potential cofactors. The products of each reaction were analysed by HPLC following DMB labelling of the sialic acid as reported previously (Monestier et al., 2017). Neu5Ac was observed as a reaction product when the enzyme was incubated with 2,7-anhydro-Neu5Ac in the presence of NAD⁺ or NADH, but not in the presence of FAD or in the absence of a cofactor (FIG. 8a).

Mass spectrometry (MS) was further used to monitor the enzymatic reaction. These analyses showed a ratio of 1:2 for 2,7-anhydro-Neu5Ac:Neu5Ac, suggesting that the reaction may be reversible. To test this further, the recombinant enzyme was incubated with Neu5Ac in the presence of NAD⁺/NADH, and the reaction products analysed by MS. The 2,7-anhydro-Neu5Ac to Neu5Ac ratio was approximately 1:2, confirming that the reaction is reversible, with Neu5Ac as the favourable product. To investigate the role of the cofactors (NAD⁺ or NADH) in the enzymatic reaction, the concentration of NADH was determined by monitoring the absorbance at 340 nm for reactions using 2,7-anhydro-Neu5Ac or Neu5Ac as substrate. No change in absorbance was detected, suggesting that the enzyme mechanism may involve oxidation and reduction of NADH cofactor. Since no net change in NADH concentration was observed during the conversion of 2,7-anhydro-Neu5Ac to Neu5Ac by RUMGNA_02695, the kinetic parameters of the enzymatic reaction were determined using the coupled reaction described above. Here, the reaction catalysed by RUMGNA 02695 was carried out in the presence of an excess of aldolase and increasing concentrations of 2,7-anhydro-Neu5Ac substrate (FIG. 8b). Using these conditions, the k_{cat} was calculated to be 0.0824±0.0043 s⁻¹ and the K_{M} 0.074±0.014 mM.

Taken together these data indicate that RUMGNA_02695 is a novel oxidoreductase required for the conversion of 2,7-anydro-Neu5Ac into Neu5Ac, which then becomes a substrate for

RgNanA. We will refer to RUMGNA_02695 as RgNanOx in the rest of the study.

### The Nan Cluster is Essential for R. gnavus to Utilise Sialoconjugates or 2,7-anhydro-Neu5Ac In Vitro

The ClosTron transformation method (Heap et al., 2010) was successfully applied to R. gnavus ATCC 29149 for the first time, enabling the generation of nan deletion mutants with an erythromycin resistance gene present in either the sense or antisense direction (relative to RgNanH). The recombination event was confirmed by PCR and the expression of the full cluster tested by qPCR. The expression of the genes flanking the cluster RUMGNA_02690 and 02702 showed levels comparable to the wild-type strain, as also observed for the first three genes of the nan cluster, RUMGNA_02701-02699, however, the nan cluster genes RUMGNA_02698-02691 showed significantly reduced expression compared to the wild-type strain.

To assess the effect of the nan cluster on the ability of R. gnavus to utilise sialic acid and sialoconjugates in vitro, R. gnavus ATCC 29149 wild-type and mutant strains were grown anaerobically with 3'SL or 2,7-anhydro-Neu5Ac. R. gnavus wild-type strain was able to utilise both 3'SL and 2,7-anhydro-Neu5Ac as a sole carbon source, but no growth was detected using the nan deletion mutants on these substrates (FIG. 9), demonstrating the importance of the nan cluster to support growth of R. gnavus ATCC 29149 on these sialic acid derivatives.

### In Vivo Colonisation of Germ-Free Mice by R. gnavus Wild-Type and Nan Mutants

To assess the impact of the nan cluster on the fitness of R. gnavus in vivo, germ-free C57BL/6J mice were gavaged with 1×10⁸ CFU R. gnavus ATCC 29149 or R. gnavus antisense nan deletion mutant or a mixture of wild-type and nan mutant strains at 1×10⁸ CFU each (FIG. 10). During mono-colonisation experiments, both strains were detectable in the faecal content at day 3, 7 and 14 post-gavage at mean levels of between 1×10⁶ and 1×10⁷ bacteria per mg of material (FIG. 10a). Both strains were also detected in the caecal content of mono-colonised mice sacrificed at day 14. The absence of the nan cluster did not affect the mouse expression response, as shown by RNA seq. In competition experiments, primers based on the insertion in the RgNanH gene were used to distinguish between wild-type and nan mutant, the wild-type strain reached mean colonisation levels comparable to the levels obtained during mono-colonisation, whereas the mutant strain was severely outcompeted, reaching only 2×10⁴ copies per mg at day 3, before decreasing further at day 7 and day 14 to levels below the level of detection, in both the faecal and caecal contents (FIG. 10b)

The impact of the nan deletion on the location of R. gnavus within the mucus layer was determined in mono-colonised mice by measuring the distance of the nan mutant or wild-type R. gnavus strains to the epithelial layer throughout the colon by fluorescent in situ hybridization (FISH) staining using confocal microscopy. The data showed that the nan mutant resided 19.70 μm from the epithelial layer, 5.06 μm further away than the wild-type strain, 14.64 μm (FIG. 10c&d).

### Bioinformatics Search for Predicted Homologous Nan Clusters

Since the success of the R. gnavus niche competition strategy depends on the organism's ability to exclusively utilize 2,7-anhydro-Neu5Ac, we searched the database for predicted homologous nan clusters to estimate how widely distributed this strategy is among bacterial isolates. MultiGeneBlast analysis revealed that predicted homologs of the R. gnavus nan cluster are shared by a limited number of species, including 37 homologous clusters in Streptococcus pneumoniae isolates (illustrated in FIG. 11 by the functionally characterized NanB from S. pneumoniae D39, Manco et al., 2006), S. suis A7, Blautia hansenii DSM 20583, Blautia sp. YL58 and Intestinimonas butyriciproducens AF211 (FIG. 11). This is also in line with the SSN bioinformatics analysis reported in FIG. 5, showing a range of species encoding NanC or IT-sialidase like genes.

In addition to the presence of a predicted IT-sialidase, the clusters share a predicted ROK family kinase, oxidoreductase, β-galactosidase, Neu5Ac lyase, and ManNAc-6-P epimerase (FIG. 11). All 37 S. pneumoniae NanB clusters share a similar organization and the more variable area between the two subclusters (white in FIG. 11) contains an additional ABC transporter compared to the other nan clusters. These Streptococcus clusters harbour a RpiR-type regulator (pink), whereas an AraC-type regulator (purple) is present in the nan clusters of the other bacterial species. Blautia sp. YL58 has the only nan cluster that contains a RUMGNA_RS11885 lipase/esterase homolog (grey), yet both the S. suis A7 and I. butyriciproducens AF211 clusters contain a different type of esterase (yellow).

A major difference between NanB/NanH IT-sialidase and NanC sialidase cluster types is the associated transporter class, a carbohydrate ABC transporter for NanB/NanH (jade green) as opposed to a sodium:solute symporter in NanC clusters (Xu et al., 2011), which may indicate a difference in the form of sialic acid being transported. Altogether, these analyses support the specialisation of the R. gnavus nan cluster, conferring the bacteria with a unique advantage over other members of the gut microbiota to colonise the mucus niche in the human colon.

1. A method of identifying, monitoring and/or diagnosing mucosal bacterial presence or infection, said method including the step of detecting at least part of a sialic acid transporter protein encoded by Ruminococcus gnavus (R. gnavus) ATCC 29149 Nan cluster.

2. A method according to claim 1 wherein the transporter protein is specific to 2,7-anhydro-Neu5Ac.

3. A method according to claim 1 wherein the substrate or solute binding protein of the ATCC 29149 Nan cluster is encoded by RUMGNA_02698.

4. A method according to claim 1 wherein the transporter protein is used as an indicator or biomarker for inflammatory bowel disease.

5. A method according to claim 4 wherein the transporter protein is used as a faecal biomarker.

6. A method according to claim 1 wherein the presence of the transporter protein is used as an indicator of likelihood of success of microbiome-targeted therapies

7. A method according to claim 6 wherein the therapy is faecal microbiota transplantation.

8. A method according to claim 1 wherein polymerase chain reaction (PCR) is used to amplify the protein and/or identify the presence of the transporter protein.

9. A method according to claim 8 wherein quantitative polymerase chain reaction (qPCR) is used to identify the presence of the transporter protein.

10. A method according to claim 1 wherein the presence or absence of the transporter protein is used to distinguish or diagnose Ulcerative Colitis or Crohn's Disease.

11. A method of inhibition of the growth of bacterium, said method including the step of inhibition of a sialic acid transporter protein.

12. A method according to claim 11 wherein the bacterium is Ruminococcus gnavus, Blautia obeum and/or Streptococcus pneumoniae.

13. A method according to claim 1 wherein the bacterium is R. gnavus.

14. A method according to claim 1 wherein Typically the transporter protein is encoded by ATCC 29149 Nan cluster.

15. A method according to claim 1 wherein the transporter protein is specific to 2,7-anhydro-Neu5Ac.

16. A method according to claim 1 wherein the substrate or solute binding protein of the ATCC 29149 Nan cluster is encoded by RUMGNA_02698.

17. A method of treatment of a mucosal disease in a subject comprising administering a therapeutically effective amount of a transport protein inhibitor.

18. A method according to claim 17 wherein the transporter protein is specific to 2,7-anhydro-Neu5Ac.

19. A method according to claim 18 wherein the inhibition is by direct or indirect inhibition.

20. A biomarker comprising RgSBP or RgOx, or one or more of the whole cluster Nan of genes.
